(19) United States
(12) Patent Application Publication
(10) Pub. No.: US 2010/0095970 A1
(43) Pub. Date: Apr. 22, 2010

(54) Mouth-Closing Device for Sleep Disorder Treatments

(76) Inventors: Sheri KATZ, Decatur, GA (US); Patrick J. STRONG, Leamington (CA)

Correspondence Address: WILEY REIN LLP, 1776 K. STREET N.W., WASHINGTON, DC, 20006, US

(21) Appl. No.: 12/426,686

(22) Filed: Apr. 20, 2009

Related U.S. Application Data
(63) Continuation-in-part of application No. PCT/US2007/022187, filed on Oct. 18, 2007.
(60) Provisional application No. 60/852,413, filed on Oct. 18, 2006.

(51) Int. Cl. A61F 5/56; A61C 9/00

(52) U.S. Cl. 128/848; 433/214

(57) ABSTRACT

The invention relates to devices designed to be used in conjunction with continuous positive airway pressure devices in order to improve the performance of the positive airway pressure in treating a disorder in breathing during sleep. In one embodiment, connection points on each side of both a maxillary and mandibular bite form are connected in order to maintain a space between the teeth substantially equal to the maximum intercuspal distance or space. Used with available continuous positive airway pressure devices, the invention can substantially improve obstructive sleep apnea or similar treatments with lower failure or patient non-compliance rates.

### REFERENCE TO RELATED APPLICATION

This application is a continuation-in-part of International application PCT/US2007/022187, filed Oct. 18, 2007, the entire contents of which are hereby incorporated by reference, which claims priority benefit of U.S. provisional application 60/852,413, filed Oct. 18, 2006, the entire contents of which are hereby incorporated by reference.

### FIELD OF THE INVENTION AND INTRODUCTION

The invention relates to devices used in the treatment of obstructive sleep apnea and improved anti-snoring systems and methods comprising these devices. In a preferred embodiment, the invention provides an improved mouthpiece design that effectively maintains the mouth closed during CPAP methods and substantially reduces or avoids the mouth-leak problems associated with prior devices. In other aspects, the invention encompasses a mouthpiece that is much easier to make and use.

### DESCRIPTION OF RELATED ART AND BACKGROUND TO THE INVENTION

The treatment of obstructive sleep apnea and snoring commonly employs a continuous positive airway pressure (CPAP) device or method, which applies pressure to hold air passages open. While widely used, the method still has drawbacks and relatively high failure or patient non-compliance rates, including mouthleaks that prevent or release the desired positive airway pressure. Some devices have been proposed the ameliorate mouthleak problems. Chin straps and similar devices that attempt to keep the mouth closed from outside the mouth are cumbersome and uncomfortable. Other devices, for example those in U.S. Pat. Nos. 6,526,982 and 5,499,633, employ bite forms that join together. However, these devices can also force the mandible forward and contain bulky or uncomfortable pieces.

Thus, there is a need in the art for improved mouthpieces that reduce or eliminate mouthleaks during CPAP, are relatively comfortable to use, and are easy to make.

### SUMMARY OF THE INVENTION

In one aspect, the invention comprises a mouth-closing device that is designed to be used in conjunction with a positive airway pressure (PAP or CPAP) device or treatment. As know in the art, the CPAP devices provide continuous positive air pressure into the lungs to through the nose in order to prevent obstructive sleep apnea. Generally, headgear or a headstrap is used with CPAP devices to keep the mouth closed. However, these headstraps or headgear often promote the movement of the tongue backward down the throat, which is counter-productive to the CPAP treatment goals. The devices of the invention, on the contrary, maintain a desired bite position in the teeth, which in turn prevents the tongue from moving down the throat. In other aspects, the devices of the invention maintain a substantially closed mouth position for the CPAP device to operate without leaks and/or without applying upward pressure on the jaw, which tends to force the tongue down the throat. Thus, the invention encompasses an improved mouthpiece device that maintains a small profile in the mouth, and/or has connection points for the upper and lower jaw towards the front of the mouth that are not offset as in mandibullar repositioning mouthpieces. The connection points can be simple buttons or round-tipped rods extending from the upper and lower jaw forms that can be attached to one another by elastic bands, for example, to keep the mouth closed.

Manufacturing the device involves casting from a mold of the patient's mouth and embedding the buttons or connection points at desired points, typically towards the front of the mouth, on each side. The buttons or connection points can be placed while the case is in its wax-up stage and still mounted on an articulator, as known in the art.

In a preferred aspect of the invention, a mouth-closing device is designed to be used in conjunction with a continuous positive airway pressure (CPAP) device or treatment in order to maintain the mouth closed in a desired or particular configuration for a patient. In general, the desired configuration is where maxillary and mandibular bite forms maintain a gap in the teeth approximately equal to the maximum intercuspal space or distance. The maximum intercuspal space can be determined, as known in the art, through the use of a mold of the patients bite or through inspection. Typically, the maximum intercuspal distance is about 5 mm. Two or more connections between the maxillary and mandibular bite forms will, as known in the art, maintain the teeth in a desired position with respect to the maximum intercuspal distance. Many of the conventional or available bite forms can be used in making the device of the invention, and ready-made bite forms that allow immediate bite forms can also be adapted and used.

In another aspect, the invention comprises an obstructive sleep apnea treatment device or anti-snoring device comprising: a maxillary bite form; a mandibular bite form; an extending maxillary connection point or button located on each side of the maxillary bite form; and an extending mandibular connection point or button located on each side of the mandibular bite form. In preferred examples, the use of a rigid, hard exterior surface for each bite form provides ample structure for the extending buttons. The connection points or buttons can be held in place to keep the mouth closed by an elastic band or similar loop. Also, the exterior portion of the connection point or button can be contoured to be smooth and small in profile and yet large enough to hold an elastic band in place. In certain embodiments, the length a button extends from the surface of the bite form is less than about 5 mm, or about 2 to about 4 mm, or most preferably about 3 mm.

In designing the bite forms for a patient, the connection points or buttons are generally placed, for the maxillary arch, approximately interproximal to the cuspid and 1st bi-cuspid and located closer to the gingival margin. The connection point or button for the mandibular arch is placed approximately below the upper button and also located near the gingival margin.

The vertical gap between top and bottom bite forms can be varied. Preferably, the vertical gap is set at approximately 5 mm, but other gaps in the range of 10 mm to 5 mm, or 8 mm to 4 mm, can be selected.

One of skill in the art is familiar with the laminated layers typically used for producing the mouthpieces or bite forms of the invention. Generally, a rigid vinyl is used, such as Proform Dual, along with an ethylene acrylic is used, such as Proflex NFC Resin. However, any appropriate combinations of polymers and resins can be selected from those approved for use in humans or available to one skilled in the art. Preferably, a base material of rigid vinyl is used and an outer layer of ethylene acrylic is used. The ethylene acrylic selected should be firm enough to stably hold the buttons in their desired positions and maintain the bite form through its use, and especially while the buttons attach the upper and lower bite forms via the elastic band. Alternatively, the button can be reinforced with a methyl methacrylate to increase strength. In other embodiments, the bite forms are made of a co-polyester plastic material or other commonly known plastic used by those skilled in the dental appliance art. However, the bite forms are not limited to any specific plastic, but rather may be made of any material suitable to carrying out the invention. Preferably, the upper and lower bite forms are made of an outer layer of a methyl methacrylate, which is easily sculpted to make particular shape modifications and other fitting adjustments, such as the desired vertical gap spacing.

Other aspects and features of the invention will become apparent from the following description, but the invention is not limited in its scope to any particular drawing or embodiment disclosed here.

### BRIEF DESCRIPTION OF THE DRAWINGS

FIG. 1 is a perspective view of a maxillary mouthpiece (bite form) in accordance with the invention, with a button or connection point at (1), showing the mouthpeice fitting over the teeth (upward arrows).

FIG. 2 is a similar view of the mandibular mouthpiece (bite form) in accordance with the invention, where button or connection point is shown at (2). The mouthpiece fits over the lower jaw (downward arrows).

FIG. 3 is a view of the maxillary mouthpiece and mandibular mouthpiece placed over the respective jaws and arranged to allow the buttons or connection points to be attached via an elastic band (not shown).

FIG. 4 represents the elastic band that fits over the buttons on each side.

FIG. 5 depicts an optional cover or lip shield that can be associated with the upper jaw buttons to further protect the mouth from opening and prevent mouthleaks laterally or from the buccal corridor.

FIG. 6 is a cut-away view of the human jaw with maxillary and mandibular mouthpieces placed over the teeth and an elastic band (3) connecting the upper (2) button with the lower (1) button. The vertical gap (5) can be set at a desired distance through the use of protrusions or features on the outer surface of one of more of the bite forms. In general, gap (5) is set to approximately the maximum intercuspal distance in a patient.

FIG. 7 is the same view as in FIG. 6, but the cover of FIG. 5 is attached to the upper buttons.

FIG. 8 depicts a view of the prior art device, with telescopic rod 63a connecting the top and bottom mouthpieces, which serves as a mandibular adjustable device.

FIG. 9 depicts the placement of the buttons or connection points in the devices of the invention.

FIG. 10 depicts an exemplary button for use in the invention. The rounded tip (9) extends out from the outer surface of the bite form and can contact the interior wall of the cheek. Other forms can be used, but preferably a smooth surface is incorporated into the tip end. The extending rod portion (8) of the button can be used to engage the elastic band, and is usually about 2 mm in length. The length of the entire button can vary, but generally an extension length from the surface of the bite form or mouthpiece is about 3 mm, or less than 5 mm. The base area of the button (note separately shown) that is adhered to the surface of the bite form during its set-up can include concave or textured features to improve the connection between the button and the bite form.

FIG. 11 shows a photograph of the upper and lower bite forms in use, with an elastic band visible and connecting the buttons associated with each bite form.

FIG. 12 shows a photograph of the bite forms used in conjunction with the lip shield as depicted in FIGS. 5 and 7. Here, the lip shield connects only at the upper bite form.

FIG. 13 depicts the situation in a typical obstructive sleep apnea case, where the airway is severally narrowed and prone to blockage.

FIG. 14 depicts the airway situation when the methods and devices of the invention are employed (device not shown). As depicted, the separation of the mandibular and maxillary jaw at a distance approximating the maximum intercuspal space prevents the tongue from sliding back and down the throat into an obstructive position. In comparing FIG. 14 to FIG. 13, the gap in the teeth (10) is evident as well as the improved airway (11) in FIG. 14., which is the situation when the inventive device is used.

FIG. 15 depicts summary data showing the response to treatment in a patient group. The respiratory disturbance index (RDI) for six patients (mean±1 SD) is plotted for three different uses of CPAP. The “CPAP alone” treatment index was 16.1±9.66. However, a significant reduction in RDI occurs when the intraoral mouth closing device (+MCD) of the invention is worn in conjunction with the CPAP (from 16.1±9.66 to 8.72±7.81, p=0.033, paired samples t-test). There is no significant difference between RDI values recorded when the CPAP is used alone versus when the CPAP with elastic chin strap (+CS) is worn simultaneously (16.1±9.66 versus 15.5±10.0, p=0.919).

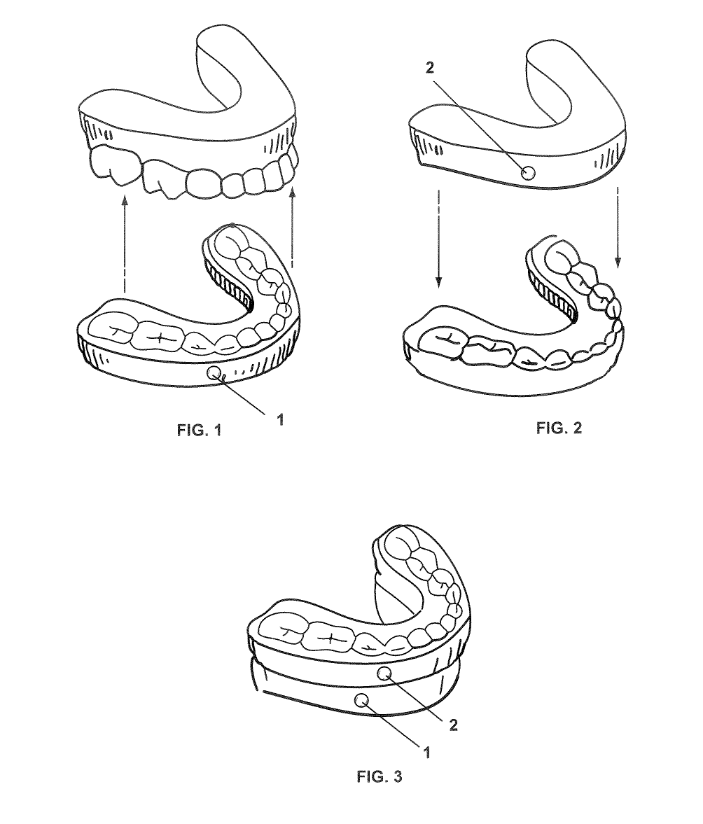

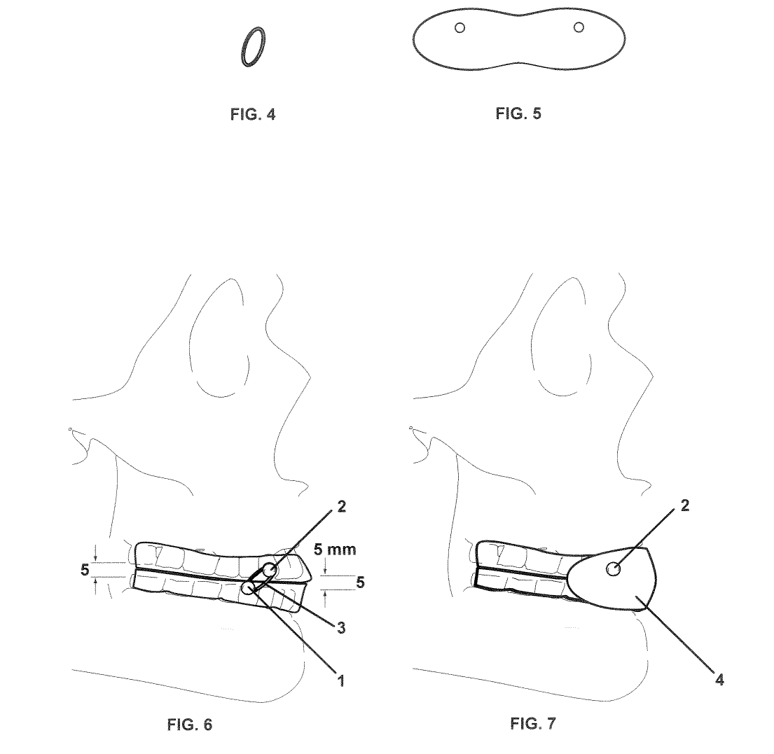

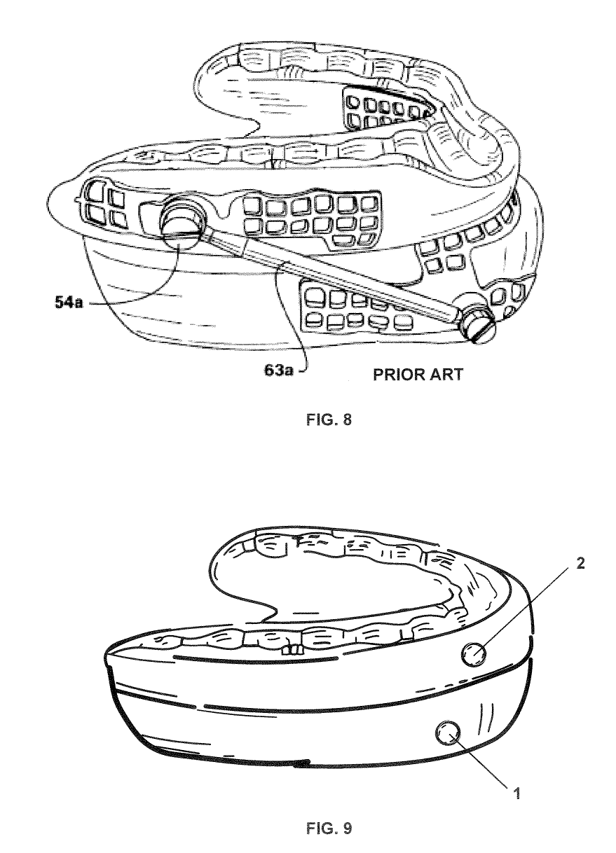

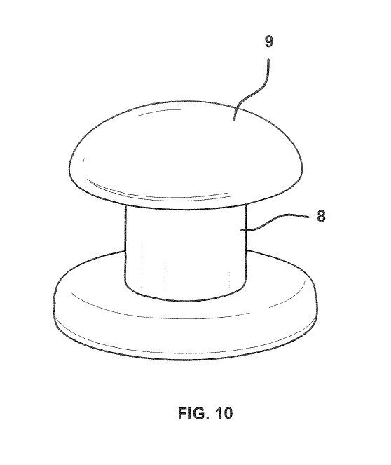

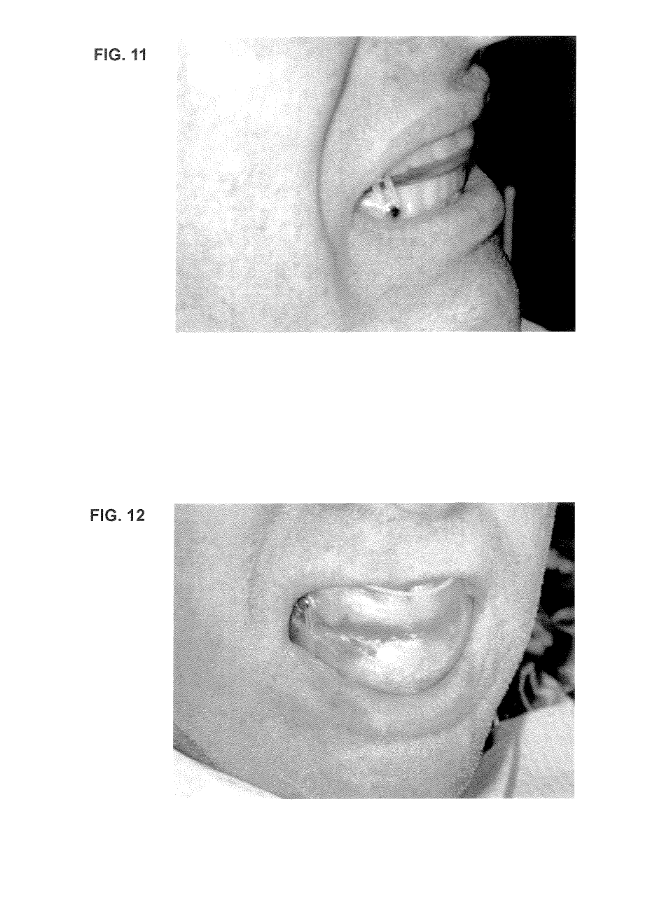

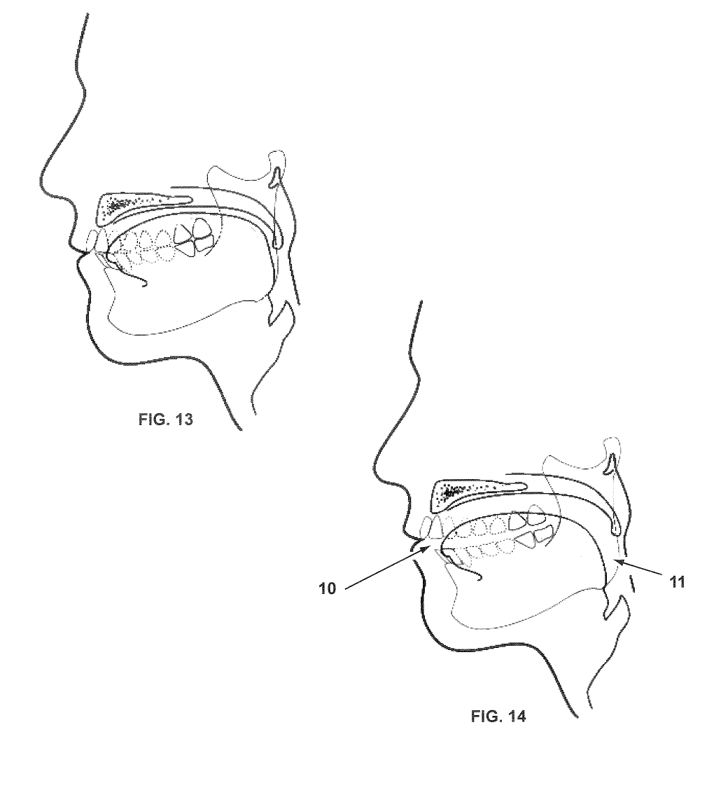

### DETAILED DESCRIPTION OF EXEMPLARY EMBODIMENTS

In making and using aspects and embodiments of this invention, one skilled in the art may employ conventional techniques and components available to one skilled in the art. Furthermore, one of skill in the art may rely upon and use documents and available commercial or research products to make and use aspects of this invention, including the selection of polymer layers of the bite forms. Patent and other documents referred to in this disclosure are specifically incorporated herein by reference and the teachings therein can be used for the purpose of making certain embodiments of the invention.

To manufacture the device of the invention, impressions are taken of a patient's dentition and maxillary and mandibular master casts are fabricated from these impressions. The master casts are then mounted to an articulator, which is adjusted to establish the proper closed position of the two master casts. As will be appreciated by those skilled in the art, this may be accomplished with proper bite registration techniques, which allows the determination of the proper, predetermined position of the mandibular cast with respect to the maxillary cast and the minimum vertical spacing between the casts. Typically, a George gauge can be used for accurate bite registration. Vent holes may then be drilled into these casts. A dual laminate material may then be attached (with heat) to the dentition of the master casts such that the harder layer of the laminate is on the outside.

In a preferred embodiment, a triple-laminated appliance is constructed. The first two layers comprise a dual-laminated blank or rigid vinyl, which is pressure formed over the model of the patient's mouth. A third and outer layer of ethylene acrylic is then used by heat curing the pressure packed thermoplastic material. The buttons can be easily placed at the desired positions into this outer layer while it is setting.

In operation, a user whose dentition is mirrored by the bite forms inserts each bite form in his mouth and bites down. The user closes his mouth and attaches the top and bottom buttons on each side with an elastic band. This prevents mouthleaks and helps to alleviate snoring and sleep apnea when used with available Positive Airway Pressure or Continuous Positive Airway Pressure (CPAP or PAP) devices. The maximum intercuspal distance spacing of the bite forms described will make the use of negative pressure or suction in the oral cavity unnecessary, and there is no requirement to retain or hold the tongue in position to achieve beneficial results.

One method to determine beneficial effects in using the devices of the invention is to record the Respiratory Disturbance Index (RDI), as known in the art. The RIM can be measured in patients, for example male patients, during three separate conditions: 1) while wearing a CPAP device alone at prescribed pressure; 2) while wearing a CPAP at prescribed pressure with the simultaneous use of a chin strap; and 3) while wearing a CPAP at prescribed pressure with the simultaneous use of the intraoral mouth closure device of the invention.

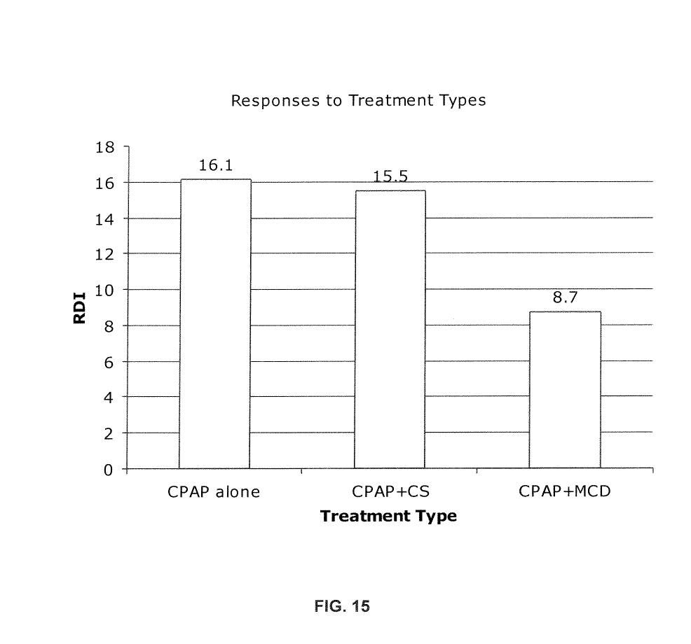

In the data of FIG. 15, the results show the RDI obtained from six patients (mean±1 SD). The first bar shows the CPAP only treatment, with an RDI of 16.1±9.66. A significant reduction in values of RDI occurs when the intraoral mouth closing device of the invention (CPAP+IOMCD) is fitted and worn in conjunction with the CPAP (from 16.1±9.66 to 8.72±7.81, p=0.033, paired samples t-test). There was no difference between RDI values recorded when the CPAP is used alone versus when the CPAP is used with the elastic chin strap (CPAP+CS) simultaneously (16.1±9.66 versus 15.5±10.0, p=0.919).

Clearly, the use of the mouth closing device of the invention improves over other available methods and devices. The mouth closing devices of the invention are also more comfortable to use with CPAP, perhaps because the vertical dimension is not altered and rather is designed to be the maximal intercuspal distance of the patient. In addition, the devices of the invention do not require the use of negative pressure in the mouth. Furthermore, the devices have an open palate and are not designed to hold, interfere with, or impede the movement of the tongue.

The connection points, or in some embodiments the buttons, as shown in the Figures are designed to be placed in positions that maintain a space substantially equal to the maximum intercuspal distance or space when the connection points between maxillary and mandibular bite forms are attached. In some circumstances, the comfort of the patient may also dictate the position or dictate some movement of the connection point placement. In addition or in the alternative, connection points of the maxillary bite form can be approximately interproximal to the cuspid and 1st bi-cuspid, and placement of the connection points on the mandibular bite form can be approximately below the maxillary connection points and not more than one tooth width away from directly below the maxillary connection point. More than one connection point may be used on each side of each bite form if desired, and the bite forms need not take the form of the buttons shown in the drawings. Multiple sized connection points or different shapes of forms of connection points can also be used. While a standard dental rubber or elastic band can generally be used to connect the mandibular and maxillary connection points, other or more rigid devices can be used. For example, wires or coated wires, springs or coated springs, and polymeric bars or rods can be used to attach the connection point, or a subset of the connection point used in two bite forms of the invention. Preferably, the devices used to attach connection points are flexible or elastic. In addition, or in the alternative, one or more connection points may be in the interior (lingual) side of the bite forms rather than all being placed on the exterior (facial) side as shown in the drawings. Accordingly, one of skill in the art has many options in positioning the connection points on the bite form in order to achieve the one or more advantages of the invention.

Any of the embodiments described or exemplified can be used in conjunction with a CPAP treatment and in methods to treat disorders in breathing during sleep. Thus, the invention specifically includes methods to improve an existing or available CPAP treatment device or the treatment of breathing disorders during sleep. Generally, the use of the devices of the invention is associated with improved CPAP results, higher satisfaction, and patient compliance levels than the prior or existing methods or devices.

The examples presented above and the contents of the application define and describe examples of the many products and methods for using them according to the invention. None of the examples and no part of the description should be taken as a limitation on the scope of the invention as a whole or of the meaning of the following claims.

What is claimed is:

1. A mouth-closing device for treating a disorder in breathing during sleep, the device used in conjunction with a positive airway pressure treatment device, the mouth-closing device comprising
a maxillary bite form;
a mandibular bite form; and
two or more connections between the maxillary and mandibular bite forms that hold the teeth of a patient to maintain the distance at occlusion between the mandibular and maxillary teeth at approximately the maximum intercuspation distance in the patient,
whereby the device prevents the tongue from occluding the airway through which the air supplied by the positive airway pressure flows.

2. The mouth-closing device of claim 1, wherein the maxillary and mandibular bite forms are made from an impression of a patient's teeth.

3. The mouth-closing device of claim 1, wherein the maxillary and mandibular bite forms are temporary molds formed on a patient's teeth.

4. The mouth-closing device of claim 2, wherein the bite forms are made from an impression where the distance at occlusion is about 5 mm.

5. The mouth-closing device of claim 1, further comprising a shield that connects to the maxillary or mandibular bite form to substantially cover the mouth and prevent airflow through the mouth.

6. An obstructive sleep apnea treatment device or anti-snoring device comprising:
a maxillary bite form;
a mandibular bite form;
an extending maxillary connection point or button located on each side of the maxillary bite form;
an extending mandibular connection point or button located on each side of the mandibular bite form,
wherein the placement of the maxillary bite form connection points or buttons is approximately interproximal to the cuspid and 1st bi-cuspid and placement of the mandibular bite form connection points or buttons is approximately below the maxillary connection points and not more than one tooth width away from directly below the maxillary connection point.

7. The device of claim 6, wherein the connection points or buttons are formed into a rounded tip rod extending from each bite form.

8. The device of claim 7, wherein the rounded tip rod extends no more than 5 mm from the surface of the bite form.

9. The device of claim 8, wherein the rounded tip rod extends no more than 3 mm from the surface of the bite form.

10. The device of claim 6, wherein the bite forms are composed of three layers, the outer layer being a heat-curing thermoplastic.

11. The device of claim 10, wherein the thermoplastic is a dental acrylic.

12. The device of claim 11, wherein the thermoplastic is a ethylene acrylic.

13. A method of forming a device of claim 1, comprising using a patient's dentition model and forming two rigid vinyl layers over the each of the maxillary and mandibular arches, which is pressure formed over the model of the patient's mouth, and then forming a third and outer layer of ethylene acrylic by heat curing a pressure packed thermoplastic material.

14. The method of claim 13, wherein the connection points or buttons are placed on the bite forms during the setting of the third or outer layer.

15. The method of claim 13, wherein the bite forms are designed to produce a vertical gap of approximately 5 mm in the mouth of the patient when using the device.

16. A method of improving a continuous positive airway pressure (CPAP) device for the treatment of breathing disorders during sleep, comprising:
providing a mouth-closing device fitted to a patients mouth having:
a maxillary bite form,
a mandibular bite form, and
two or more connections between the maxillary and mandibular bite forms that hold the teeth of the patient to maintain the distance at occlusion between the mandibular and maxillary teeth at approximately the maximum intercuspation distance in the patient;
and causing the CPAP treatment to occur on the patient,
whereby the device prevents the tongue from occluding the airway through which the air supplied by the positive airway pressure flows.

17. The method of claim 16, wherein the bite forms are designed to produce a vertical gap between the bite forms of approximately 5 mm in the mouth of the patient.

18. The method of claim 16, wherein the bite forms are designed to produce a vertical gap between the bite forms from about 5 mm to about 10 mm in the mouth of the patient.

19. The method of claim 16, wherein the bite forms are designed to produce a vertical gap between the bite forms from about 4 mm to about 8 mm in the mouth of the patient.

20. A method of treating a breathing disorder during sleep comprising fitting a maxillary bite form and a mandibular bite form for a patient's mouth, placing at least one extending maxillary button on each side of the maxillary bite form, placing at least one extending mandibular button located on each side of the mandibular bite form, wherein the placement of the maxillary bite form buttons is approximately interproximal to the cuspid and 1st bi-cuspid and placement of the mandibular bite form connection points or buttons is approximately below the maxillary connection points and not more than one tooth width away from directly below the maxillary connection point, inserting the maxillary bite form and a mandibular bite form into the patient's mouth and connecting the buttons with an elastic band, and applying positive airway pressure through the nasal passage.

21. The method of claim 20, wherein the bite forms are designed to produce a vertical gap of approximately 5 mm in the mouth of the patient.

22. The method of claim 21, wherein at least one bite form button is placed on the facial side of the bite form.
